(19) United States
(12) Patent Application Publication
(10) Pub. No.: US 2024/0067596 A1
(43) Pub. Date: Feb. 29, 2024

(54) CONTINUOUS METHOD FOR OBTAINING 2-ETHYLHEXYL ACRYLATE

(71) Applicant: BASF SE, Ludwigshafen am Rhein (DE)

(72) Inventors: Ortmund LANG, Ludwigshafen am Rhein (DE); Claus HECHLER, Ludwigshafen am Rhein (DE); Cornelis DE RUITER, Ludwigshafen am Rhein (DE); Marvin KRAMP, Ludwigshafen am Rhein (DE); Dominik HERBRECHT, Ludwigshafen am Rhein (DE)

(21) Appl. No.: 18/273,790

(22) PCT Filed: Jan. 24, 2022
(86) PCT No.: PCT/EP2022/051516

§ 371 Date: Jul. 24, 2023

(30) Foreign Application Priority Data
Jan. 25, 2021 (EP) 21153162.9

(51) Int. Cl. C07C 69/54; B01D 1/00

(52) U.S. Cl. CPC C07C 69/54; B01D 1/0088

(57) ABSTRACT

The present invention relates to a continuous process for obtaining 2-ethylhexyl acrylate (2-EHA) from a mixture (1) that is liquid under an absolute pressure in the range from 0.5 to 100 bar and has a temperature in the range from 0 to 300° C., comprising 2-EHA, at least one high boiler, at least one homogeneous catalyst, and at least one low boiler, wherein the mixture (1) is depressurized by a pressure-maintenance device (3) to an absolute pressure level in the range from 0.1 to 10 bar, wherein the resulting two-phase gas/liquid mixture (16) is continuously supplied to a helical-tube evaporator (4) in which, at a temperature in the range from 50 to 300° C., the 2-EHA content of the liquid phase of the two-phase gas/liquid mixture is reduced by partial evaporation, this being accompanied by a parallel increase in the 2-EHA content of the gas phase of the two-phase gas/liquid mixture, and the two phases are discharged in the form of a resulting two-phase gas/liquid output stream (17).

The present invention relates to a continuous process for obtaining 2-ethylhexyl acrylate (2-EHA) from a mixture (1) that is liquid under an absolute pressure in the range from 0.5 to 100 bar and has a temperature in the range from 0 to 300° C., comprising 2-EHA, at least one high boiler, at least one homogeneous catalyst, and at least one low boiler.

The production of 2-EHA is disclosed for example in DE 10246869 A1 (BASF AG). The production of (meth)acrylic esters here also gives rise to the by-product 2-EHA. In the process according to D1, the acid-catalyzed esterification of acrylic acid with the 2-ethylhexanol takes place in a homogeneous liquid phase, the esterification being carried out in a reaction zone equipped with at least one distillation unit, via which the water formed in the esterification is, together with 2-ethylhexene, 2-ethylhexanol, and 2-EHA, removed and condenses and separates into an aqueous phase and an organic phase.

DE 10246869 A1 (BASF AG) further discloses that the 2-EHA is obtained by thermally treating a residue produced from distillation of the residue. The thermal treatment is carried out by means of a discontinuous process in a stirred tank that is also referred to as a “batch process”. More particularly, the thermal treatment takes place preferably at 140 to 200° C. and an absolute pressure of 20 to 300 mbar in a stirred apparatus. This thermal treatment results in cleavage reactions, which are undesirable. The cleavage residues produced during the cleavage reactions, primarily the product of value 2-EHA, 2-ethylhexanol, acrylic acid, and a 2-ethylhexene isomer mixture, are continuously separated, condensed, and returned to the esterification in the 2-EHA production process. The cleavage residues, which are still pumpable, are disposed of and in this process incinerated, for example. These cleavage residues generally comprise 25 to 35% of esterification catalyst, 20 to 30% of the product of value 2-EHA, 10 to 20% of oxyesters, 2 to 3% of inhibitors, and 25 to 30% of high boilers. If desired, the cleavage residues can in part be recycled again to the process, to an extent of 0 to 80%. To improve the pumpability, the cleavage residues can typically be mixed with solvents such as Oxo Oil and then for example be thermally utilized. However, this approach involves more work and higher costs on account of the additional resources required. The disadvantage of this process is that, despite possible optimizations, toward the end of the batch process because of the very high concentration of homogeneous catalyst during cleavage of the high boilers it is no longer the product of value 2-EHA that is formed, but instead a 2-ethylhexene isomer mixture. These low boilers are no longer employable in the process and must be disposed of. In addition, the long residence time in the batch process results in the further formation of polymers that are no longer able to undergo cleavage and thus in a sharp increase in the viscosity of the residue. The solvent required for dilution results in additional work and higher costs and the amount of residue is also increased.

More efficient processes for obtaining or separating 2-EHA are not known.

Another process for obtaining a different product of value, namely cyclododecatriene (CDT), is disclosed in EP 1907342 B1 (BASF SE), which describes a continuous process based on a pressure-maintenance device and a helical-tube evaporator. Because of the short residence time of the solution in the helical-tube evaporator, undesired cleavage residues in the solution comprising CDT, high boilers, and other polymers are significantly reduced. Liquid and gas are separated from one other by a downstream gravity separator. The product of value CDT is then largely found in the condensate.

Helical-tube evaporators are well known and are described for example in patent application DE 19600630 A1 (Bayer AG). This discloses an evaporator apparatus in which the mechanical force necessary to keep the heat exchange surface clear is brought about not by rotating internals, but by flow forces. This evaporator apparatus consists of a single, helical tube that is heated externally. This single-tube evaporator is now operated such that the solution or suspension is fed into the apparatus in a superheated state under absolute pressure, such that a portion of the volatile constituents of the solution evaporates as soon as it enters the apparatus. This vapor takes on the role of transporting the increasingly viscous solution or suspension through the apparatus and ensures that the heat-transfer surface is kept clear.

The object was to provide a novel, more efficient process for evaporating the product of value 2-EHA from a mixture (1) that is produced for example as a reaction discharge in the production of (meth)acrylic esters by acid-catalyzed esterification of acrylic acid with 2-ethylhexanol. The production of (meth)acrylic esters can be enabled for example by the process according to DE 10246869 A1 (BASF AG). At the same time, the novel, more efficient process should also keep capital costs and outlay on plant and apparatus construction as low as possible.

Such a mixture (1) comprises 2-EHA, at least one high boiler, at least one homogeneous catalyst, and at least one low boiler.

Preferred and exemplary configurations for the mass fractions of the components present in mixture (1) are shown below in percent by weight, where the sum of the 2-EHA, high boilers, homogeneous catalyst, low boilers, and additional components comes to 100% by weight. The additional components have only a negligible effect on the process according to the invention, consequently these additional components are not of industrial relevance for the process according to the invention.

A preferred configuration for the individual components of the mixture (1) and mass fractions thereof based on the mixture (1) in percent by weight is as follows:

| | | |
|---|---|---|
| 2-EHA: | ≥10.0% | by weight |
| High boilers: | ≥0.3% | by weight |
| Polymers: | 0.1-10.0% | by weight |
| 2-Ethylhexyl 3-(2-ethylhexoxy)-propionate: | ≥0.1% | by weight |
| 2-Ethylhexyl 2-diacrylate: | 0.1-12.0% | by weight |
| Homogeneous catalyst: | 0.1-15.0% | by weight |
| Low boilers: | 0.1-20.0% | by weight |
| Water: | 0-15.0% | by weight |
| Acrylic acid: | 0-15.0% | by weight |
| 2-Ethylhexanol: | 0-15.0% | by weight |
| 2-Ethylhexene isomers: | 0-15.0% | by weight |
| Additional components: | 0-10.0% | by weight |

In a particularly preferred configuration, the individual components of the mixture (1) and mass fractions thereof based on the mixture (1) in percent by weight are as follows:

| | | |
|---|---|---|
| 2-EHA: | 20.0-80.0% | by weight |
| High boilers: | 0.3-60% | by weight |
| Polymers: | 0.1-6.0% | by weight |
| 2-Ethylhexyl 3-(2-ethylhexoxy)-propionate: | 0.1-45.0% | by weight |
| 2-Ethylhexyl 2-diacrylate: | 0.1-10.0% | by weight |
| Homogeneous catalyst: | 0.1-15.0% | by weight |
| Low boilers: | 0.1-15.0% | by weight |
| Water: | 0-10.0% | by weight |
| Acrylic acid: | 0-10.0% | by weight |
| 2-Ethylhexanol: | 0-10.0% | by weight |
| 2-Ethylhexene isomers: | 0-10.0% | by weight |
| Additional components: | 0-6.0% | by weight |

In an exemplary configuration, the individual components of the mixture (1) and mass fractions thereof based on the mixture (1) in percent by weight are as follows:

| | | |
|---|---|---|
| Water | 0.2% | by weight |
| 2-Ethylhexene isomers | 0.3% | by weight |
| Acrylic acid | 0.6% | by weight |
| 2-Ethylhexanol | 0.4% | by weight |
| 2-Ethylhexyl acrylate (2-EHA) | 84.5% | by weight |
| 2-Ethylhexyl 3-(2-ethylhexoxy)-propionate | 4.0% | by weight |
| 2-Ethylhexyl 2-diacrylate | 4.4% | by weight |
| Polymers | 4.3% | by weight |
| p-Toluenesulfonic acid as | 1.1% | by weight |
| homogeneous catalyst | | |
| Additional components | 0.2% | by weight |

In a further exemplary configuration, the individual components of the mixture (1) and mass fractions thereof based on the mixture (1) in percent by weight are as follows:

| | | |
|---|---|---|
| Water | 4.0% | by weight |
| 2-Ethylhexene isomers | 4.5% | by weight |
| Acrylic acid | 4.1% | by weight |
| 2-Ethylhexanol | 6.0% | by weight |
| 2-Ethylhexyl acrylate (2-EHA) | 10.7% | by weight |
| 2-Ethylhexyl 3-(2-ethylhexoxy)-propionate | 50.0% | by weight |
| 2-Ethylhexyl 2-diacrylate | 10.0% | by weight |
| Polymers | 10.0% | by weight |
| p-Toluenesulfonic acid as | 0.5% | by weight |
| homogeneous catalyst | | |
| Additional components | 0.2% | by weight |

In a further exemplary configuration, the individual components of the mixture (1) and mass fractions thereof based on the mixture (1) in percent by weight are as follows:

| | | |
|---|---|---|
| Water | 0.7% | by weight |
| 2-Ethylhexene isomers | 1.1% | by weight |
| Acrylic acid | 0.4% | by weight |
| 2-Ethylhexanol | 0.5% | by weight |
| 2-Ethylhexyl acrylate (2-EHA) | 12.4% | by weight |
| 2-Ethylhexyl 3-(2-ethylhexoxy)-propionate | 79.8% | by weight |
| 2-Ethylhexyl 2-diacrylate | 3.0% | by weight |
| Polymers | 1.0% | by weight |
| p-Toluenesulfonic acid as | 0.8% | by weight |
| homogeneous catalyst | | |
| Additional components | 0.3% | by weight |

In a further exemplary configuration, the individual components of the mixture (1) and mass fractions thereof based on the mixture (1) in percent by weight are as follows:

| | | |
|---|---|---|
| Water | 0.8% | by weight |
| 2-Ethylhexene isomers | 1.0% | by weight |
| Acrylic acid | 0.5% | by weight |
| 2-Ethylhexanol | 0.4% | by weight |
| 2-Ethylhexyl acrylate (2-EHA) | 60.3% | by weight |
| 2-Ethylhexyl 3-(2-ethylhexoxy)-propionate | 11.0% | by weight |
| 2-Ethylhexyl 2-diacrylate | 0.4% | by weight |
| Polymers | 1.6% | by weight |
| p-Toluenesulfonic acid as | 15.0% | by weight |
| homogeneous catalyst | | |
| Additional components | 9.0% | by weight |

In a further exemplary configuration, the individual components of the mixture (1) and mass fractions thereof based on the mixture (1) in percent by weight are as follows:

| | | |
|---|---|---|
| Water | 0.4% | by weight |
| 2-Ethylhexene isomers | 0.1% | by weight |
| Acrylic acid | 0.4% | by weight |
| 2-Ethylhexanol | 0.9% | by weight |
| 2-Ethylhexyl acrylate (2-EHA) | 52.5% | by weight |
| 2-Ethylhexyl 3-(2-ethylhexoxy)-propionate | 28.8% | by weight |
| 2-Ethylhexyl 2-diacrylate | 5.8% | by weight |
| Polymers | 3.0% | by weight |
| p-Toluenesulfonic acid as | 4.1% | by weight |
| homogeneous catalyst | | |
| Additional components | 4.0% | by weight |

Under the same pressure, for example standard pressure, the low boilers have a lower boiling temperature than 2-EHA and the high boilers have a higher boiling temperature than 2-EHA. The boiling point at standard pressure is 218° C. for 2-EHA. Under standard pressure, the low boilers are generally in a range from 50 to 215° C. and the high boilers in a range from 220 to 400° C.

The novel process should avoid or at least significantly reduce the formation of cleavage residues and also the formation of polymers, since these phenomena result in excessively high viscosity in the residue, thereby making the process much more laborious.

In addition, this process should produce a discharge from the reaction of 2-EHA per kilogram similar to that of a batch process, for example the process described in DE 10246869 A1 (BASF AG), and deliver the same or improved quality in respect of color, color stability, odor and/or purity. Furthermore, losses of the product of value 2-EHA due to residual contents in the bottoms discharge and to the formation of low boilers (for example 2-ethylhexene isomers) and high boilers (for example polymers) must also be minimized. This also makes the process less energy-intensive.

These objects were achieved in accordance with the invention by a continuous process for obtaining 2-ethylhexyl acrylate (2-EHA) from a mixture (1) that is liquid under an absolute pressure in the range from 0.5 to 100 bar and has a temperature in the range from 0 to 300° C., comprising 2-EHA, at least one high boiler, at least one homogeneous catalyst, and at least one low boiler, which is characterized in that the mixture (1) is depressurized by a pressure-maintenance device (3) to an absolute pressure level in the range from 0.1 to 10 bar, wherein the resulting two-phase gas/liquid mixture (16) is continuously supplied to a helical-tube evaporator (4) in which, at a temperature in the range from 50 to 300° C., the 2-EHA content of the liquid phase of the two-phase gas/liquid mixture is reduced by partial evaporation, this being accompanied by a parallel increase in the 2-EHA content of the gas phase of the two-phase gas/liquid mixture, and the two phases are discharged in the form of a resulting two-phase gas/liquid output stream (17).

The invention further relates to preferred configurations of the process according to claims 2 to 18.

It was found that in a continuous process with short residence times, for example in the range from 0.3 to 10 minutes, the formation of low boilers from 2-ethylhexene isomers and the formation of high-boiling polymers can be largely prevented. This means that the cleavage residues can be prevented or at least significantly reduced.

It was also found that the process according to the invention should be carried out not just with the shortest possible residence time, but also at low temperature and low absolute pressure.

This could accordingly be achieved using a thin-film evaporator or short-path evaporator, optionally in combination with an upstream falling-film evaporator, forced-circulation evaporator or forced-circulation flash evaporator. Thin-film evaporators or short-path evaporators are described inter alia in the dissertation cited below, on pages 44 to 46:

M. Dippel, Entwicklung einer Methode zur Ermittlung produktschonender Betriebs- und Design-parameter von Wärmeübertragerrohren für temperaturempfindliche Prozessstrome [Development of a method for determining product-conserving operating and design parameters for heat-exchanger tubes for temperature-sensitive process streams], Faculty of Mechanical Engineering of the Ruhr University Bochum, 2016.

The process is however found to be technically complex on account of the apparatus employed for this purpose. A further drawback of this apparatus concept is the comparatively high capital costs for the combination of falling-film evaporator and thin-film evaporator and the high variable costs for operating the thin-film evaporator. Furthermore, the use of evaporator types such as falling-film evaporators, forced-circulation evaporators, and forced-circulation flash evaporators is associated with considerable process risks, since the high-boiling components present in a feed stream and the decomposition products that can occur during evaporation tend to form deposits on hot surfaces. In addition, deposits can also form in thin-film evaporators, for example on the internal wiper system, which can lead to system outages.

It was found that high boilers can in accordance with the invention be removed in an apparatus of comparatively simple construction—the helical-tube evaporator (4)—without external mixing of the liquid film and with avoidance of deposit formation on the heated walls. This would not have been anticipated by those skilled in the art, since helical-tube evaporators have significantly greater heat flow densities compared to conventional thin-film evaporators and are as a result run at significantly greater temperature differentials, which typically results in increased formation of polymers and deposits.

Although very little additional product of value 2-EHA is produced in the process according to the invention, or none at all, the overall process reconciliation shows that the process according to the invention affords a yield of 2-EHA similar to that of a batch process, such as the batch process according to DE 10246869 A1 (BASF AG).

Because of the prevention of the formation of high boilers in the process according to the invention, the residue (10) obtained remains pumpable even without a diluent.

In the process according to the invention, the short residence time of the two-phase gas/liquid mixture (16) in the helical-tube evaporator (4) means that the formation of polymers due to excessive thermal stress is effectively prevented or at least significantly reduced compared to a batch process as mentioned above. The temperatures in the helical-tube evaporator (4) are here in the range from 50 to 300° C., preferably in the range from 100 to 200° C., and more preferably in the range from 140 to 160° C.

Contrary to previous experience with conventional evaporator concepts, losses of 2-EHA due to polymer formation in the evaporator system thus remain very low, in the preferred case less than 1% by weight based on the mixture (1).

A novel solution for obtaining 2-EHA in an efficient process is thus provided that, in addition to low outlay on apparatus, permits long service lives and low operating costs.

In an advantageous embodiment of the process, a preheater (2) upstream of the pressure-maintenance device (3) heats the liquid mixture (1) to a temperature in the range from 100 to 200° C., if the mixture (1) does not have a temperature of at least 100° C.

This avoids effects such as soiling and/or caking, since the mixture (1) has an elevated temperature, and thus a lower viscosity, from the outset.

In a preferred embodiment, the helical-tube evaporator (4) is operated at an absolute pressure in the range from 1 to 2000 mbar.

In a further preferred configuration, the proportion of 2-EHA in the liquid phase is in a single pass through the helical-tube evaporator (4) reduced to a 2-EHA content of less than 20% by weight.

In a particularly preferred configuration, the proportion of 2-EHA in the liquid phase is in a single pass through the helical-tube evaporator (4) reduced to a 2-EHA content of less than 10% by weight.

This is made possible by the use according to the invention of the helical-tube evaporator (4) and by process parameters such as the temperature of the mixture (1) on exiting the preheater (2). This results in an efficient process in which 2-EHA losses in the residue (10) are largely avoided.

In a preferred embodiment, the formation of 2-ethylhexene isomers in the process is less than 2% by weight based on the mixture (1). This is made possible inter alia by a short residence time and/or a low temperature in the helical-tube evaporator.

It is preferably also possible to return part of the liquid phase of the two-phase gas/liquid output stream (17) withdrawn from the helical-tube evaporator (4) back to the helical-tube evaporator (4) for further partial evaporation. This can further improve the purification of the distillate (9). Depending on the associated costs and the composition of the mixture, it is also possible to achieve almost complete separation of 2-EHA from the mixture (1).

In a further embodiment, a stripping gas (7) can be added to the two-phase gas/liquid mixture (16) downstream of the pressure-maintenance device (3), for example through a supply conduit, so that the partial evaporation in the helical-tube evaporator (4) is carried out in the presence of a stripping gas (7). The stripping gas (7) can preferably be steam or an inert gas, preferably nitrogen, or a mixture of different gases, which lowers the partial pressure of the vaporizable components in the mixture (1) and increases the gas velocity.

Preferably, the supply of stripping gas (7) can be, in order to achieve a preferred flow pattern in the helical-tube evaporator (4) and/or to adjust the residence time of the two-phase gas/liquid mixture (16) in the helical-tube evaporator (4). In addition, residual low boilers can be removed from the gas/liquid mixture (16) by stripping. The amount of stripping gas to the helical-tube evaporator (4), based in each case on the mixture (1), is preferably in the range from greater than 0% to 50% by weight, particularly preferably in the range from greater than 0% to 20% by weight, and very particularly preferably in the range from greater than 0% to 5% by weight. What is thus referred to as the total feed stream comprises the mixture (1) and the stripping gas (7).

The stripping gas (7) can also preferably be loaded with low boilers, thereby allowing better separation of the low boilers in the helical-tube evaporator.

The residence time can generally be defined by the flow rate and by the geometry of the helical-tube evaporator (4), which has a helical tube (5). The residence time in the helical-tube evaporator (4) and the associated pipework system is preferably set in the range from 0.3 to 10 minutes, more preferably in the range from 0.5 to 2 minutes. In particular, this reduces thermal decomposition (cleavage reaction) of the target product and polymer formation, or even avoids it altogether.

The process is generally carried out continuously, but the separation can principle also be carried out as a continuous batchwise process.

Under particular circumstances it may also be advisable to fin the helical tube (5) in the helical-tube evaporator (4) on the inside and/or outside. This is understood as meaning the attachment of fins to the inside or outside of the helical tube (5). These fins improve the performance of the helical tube (5). This improvement is brought about both through providing a larger heat-transfer surface area and by creating additional turbulence. The inside of the helical tube (5) may also be completely or partially equipped with wire knits. This is understood as meaning the introduction of wire knits into the helical tube (5), which improves heat transfer and mass transfer.

In a further embodiment it is possible that, instead of a single helical-tube evaporator (4), two or more helical-tube evaporators (4) are connected in series to form an evaporator cascade, wherein the gas/liquid mixture (16) flowing into the evaporator cascade undergoes a gradual reduction in the 2-EHA content of its liquid phase through partial evaporation of the liquid phase. In this variant, it can be advantageous to operate the individual helical-tube evaporators of the evaporator cascade at different or identical pressures, preferably in the range from 1 to 2000 mbar and more preferably in the range from 5 to 200 mbar.

In a further embodiment it is possible that, instead of a single helical-tube evaporator (4), two or more helical-tube evaporators (4) are connected in parallel to form an evaporator cascade, wherein the gas/liquid mixture (16) flowing into the evaporator cascade undergoes a reduction —split between the two evaporators—in the 2-EHA content of its liquid phase through partial evaporation of the liquid phase.

In this variant, it can be advantageous to operate the individual helical-tube evaporators of the evaporator cascade at different or identical pressures, preferably in the range from 1 to 2000 mbar and more preferably in the range from 5 to 200 mbar.

In a further embodiment, an evaporator stage (each individual stage in each case represents an individual helical-tube evaporator) of the evaporator cascade can optionally also be operated at least partially with heat integration.

Heat integration of, for example, two helical-tube evaporators (4) can preferably be designed as follows:

A first helical-tube evaporator (4) is operated at a product-side absolute pressure of 200 mbar and heated with 17 bar (abs.) of heating steam (approx. 204° C.). The steam condensate accumulating in the first helical-tube evaporator (4) at a temperature of, for example, 150° C. is used to heat the second helical-tube evaporator (4), which is operated at 50 mbar. This has the advantage of consuming less steam.

By appropriately setting the operating point of the helical-tube evaporator, very high area-specific performance is achieved with short residence times.

Thus, in laboratory tests up to 5 kg/h of a 2-EHA-containing solution was able to flow through a helical tube having an internal diameter of 6 mm without problem.

In a further embodiment, the two-phase gas/liquid output stream (17) from the helical-tube evaporator (4) is supplied to a downstream separator (6), which is preferably a gravity separator.

The gravity separator is here preferably operated at an absolute pressure in the range from 1 to 2000 mbar, preferably at an absolute pressure in the range from 5 to 200 mbar, and more preferably in the range from 15 to 50 mbar.

In principle, a centrifugal droplet separator or a separator with a demister could also be used instead of a gravity separator. All these separators have the function of separating liquid from vapor/gas.

The evaporation rate is understood as meaning the ratio of the amount of distillate to the feed rate. The evaporation rate can be determined for example by experiments.

The evaporation rate of the two-phase gas/liquid mixture (16) in the helical-tube evaporator (4) also determines the concentration of the product of value 2-EHA in the bottoms product, the bottoms product being the product that collects in the bottoms region of a downstream separator (6). The separator (6) is preferably a gravity separator.

The setting of the heating temperature and of the pressure in the helical-tube evaporator (4) determines the evaporation rate of the two-phase gas/liquid mixture (16).

The absolute pressure downstream of the pressure-maintenance device (3) may vary greatly during operation: in the process according to the invention it is in the range from 0.1 to 10 bar. The absolute pressure establishes itself according to the operating parameters. The absolute pressure in the separator (6) is set in the range from 1 to 2000 mbar, preferably in the range from 5 to 200 mbar, and more preferably in the range from 15 to 50 mbar.

The pressure downstream of the pressure-maintenance device (3) depends inter alia on the following parameters:
Absolute pressure in the separator (6)
Length and diameter of the helical tube (5)
Material properties such as the density or the viscosity of the liquid mixture (1)
Temperature downstream of the preheater (2)
Mass flow and volume flow through the helical tube (5) of the helical-tube evaporator (4)

In a preferred embodiment, the formation of polymers in the helical-tube evaporator (4) and in the separator (6) is together less than 5% by weight based on the mixture (1). This is made possible inter alia by a short residence time and/or a low temperature in the helical-tube evaporator (4).

In a preferred embodiment, the gaseous fraction of the two-phase gas/liquid output stream (17) supplied to the separator (6) is supplied from the separator (6) to a condenser (12) and condensed in the condenser (12) to form a distillate (9). This gaseous fraction is also referred to as the vapor stream.

A vapor stream can be condensed into a distillate (9) in conventional condensers (12) such as shell-and-tube apparatuses or quench condensers.

The resulting condensates, which essentially comprise the product of value 2-EHA, can be worked up in conventional distillation units or used further directly. The concentration of 2-EHA in the distillate (9) is normally between 30% and 90% by weight.

The bottoms stream from the separator (6) essentially comprises the high boilers formed during the reaction and catalyst fractions. Depending on the mode of operation, the content of the product of value 2-EHA in the bottoms stream is less than 30% by weight, preferably less than 10% by weight, more preferably less than 5% by weight. In a specific embodiment, residual proportions of 2-EHA of even less than 1% by weight can be achieved.

In a further embodiment, the absolute pressure in the vapor stream is set at 1 to 104 mbar, preferably 1 to 10³ mbar, more preferably 1 to 200 mbar. In a further preferred embodiment, the vapor stream is at an absolute pressure in the range from 1 to 100 mbar.

Through an appropriate design of the geometry of the helical-tube evaporator (4), and of the helical tube (5) thereof in particular, it is possible in a preferred embodiment for a wavy film flow, in the sense of a turbulent flow, to be established in the pipe, depending on the overall volume flow rate, the gas fraction, the requisite absolute pressure in the separator (6), etc. This achieves intensive heat transfer and mass transfer. The high throughputs result in high wall shear stresses, thereby effectively preventing the buildup of caked deposits on the heated walls. The helical-tube evaporator (4) may be heated for example by means of condensing steam or with the aid of a thermostated oil circuit. Electrical heating is also possible.

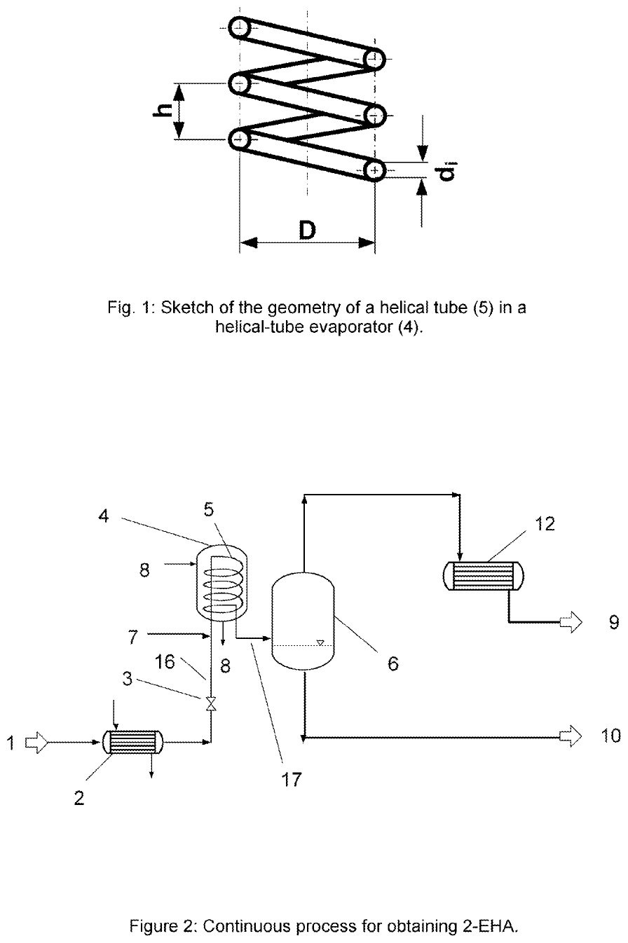

A preferred geometry for the helical-tube evaporator (4) is shown in FIG. 1. In the figure, the parameter dᵢ is the internal diameter of the tube, D is the diameter of curvature of the helical tube (5) (also referred to as the diameter of the helical coil), and h is the pitch of the helical tube (5).

The dimensionless ratio of curvature a is the ratio between the internal diameter dᵢ and the diameter of curvature D and is represented by the formula:

a=dᵢ/D

The dimensionless pitch b is the ratio between the pitch of the helical tube h and the diameter of curvature D and is represented by the formula:

b=h/D

The dimensionless ratio of curvature a is in the range from 0.01 to 0.5, preferably in the range from 0.01 to 0.4, more preferably in the range from 0.02 to 0.2, and most preferably in the range from 0.02 to 0.1.

The dimensionless pitch b is in the range from 0.01 to 1.0, preferably in the range from 0.02 to 0.8, more preferably in the range from 0.05 to 0.5, and most preferably in the range from 0.06 to 0.18.

The dimensionless pitch b is here to be set independently of the dimensionless ratio of curvature a.

Thus, a helical tube (5) in the helical-tube evaporator (4), or each individual helical tube of a helical-tube evaporator in the case of an evaporator cascade, should independently have a dimensionless ratio of curvature a in the range from 0.01 to 0.5 and a dimensionless pitch b in the range from 0.01 to 1.0.

Preferably, a helical tube (5) in the helical-tube evaporator (4), or each individual helical tube of a helical-tube evaporator in the case of an evaporator cascade, should independently have a dimensionless ratio of curvature a in the range from 0.01 to 0.4 and a dimensionless pitch b in the range from 0.02 to 0.8.

Particularly preferably, a helical tube (5) in the helical-tube evaporator (4), or each individual helical tube of a helical-tube evaporator in the case of an evaporator cascade, should independently have a dimensionless ratio of curvature a in the range from 0.02 to 0.1 and a dimensionless pitch b in the range from 0.06 to 0.18.

In the case of evaporator cascades, the design of the helical tube as determined inter alia by the ratio of curvature a or by the dimensionless pitch b applies to all helical-tube evaporators.

The parameters for the individual helical tube can be set independently for each individual helical-tube evaporator.

The invention will be discussed in more detail below with reference to the drawings. The drawings are to be understood as diagrammatic illustrations. They do not constitute a limitation of the invention, for example with regard to specific dimensions or design variants. In the figures:

FIG. 1 shows a sketch of the geometry of a helical tube (5) in a helical-tube evaporator (4). The pitch h, the internal diameter dᵢ, and the diameter (of curvature) D of the helical tube (5) are shown.

FIG. 2 shows a diagram of a continuous process according to the invention for obtaining 2-EHA in which the separation of inter alia high boilers is carried out in a continuous helical-tube evaporator system.

A liquid mixture (1) is supplied to a preheater (2), then depressurized via a pressure-maintenance device (3) and supplied to a helical-tube evaporator (4) in the form of a two-phase gas/liquid mixture (16). The distillate (9) to be condensed via a condenser (12) is separated from a residue (10) by means of a separator (6). Optionally, the distillate (9) can be supplied to the mixture (1) upstream of the preheater (2) so as to be able to concentrate the target product 2-EHA.

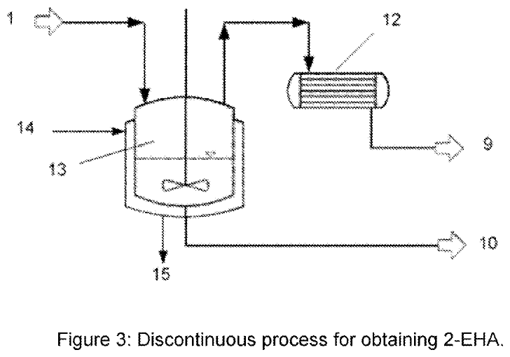

FIG. 3 shows a diagram of a discontinuous prior art process for obtaining 2-EHA. A mixture (1) is supplied to a discontinuously operated stirred tank (13). The separation of inter alia high boilers takes place in the stirred tank (13) with external heating, wherein the heating may be effected by heating steam (14) and the condensate (15) resulting therefrom is discharged from the stirred tank (13). A residue (10) is discharged from the stirred tank (13). A vapor stream is passed from the stirred tank (13) into a condenser (12), in which the vapor stream condenses. The distillate (9) containing the target product 2-EHA can optionally be recycled back to the process.

### LIST OF REFERENCE NUMBERS USED

1 Mixture
2 Preheater
3 Pressure-maintenance device
4 Helical-tube evaporator
5 Helical tube
6 Separator
7 Stripping gas
8 Heating oil
9 Distillate
10 Residue
12 Condenser
13 Stirred tank
14 Heating steam
15 Condensate
16 Two-phase gas/liquid mixture
17 Output stream

### EXAMPLES

### Example 1

Example 1 discloses a continuous process configuration according to the invention, which is shown in FIG. 2. In this configuration, the high boilers are separated in a continuous helical-tube evaporator system.

The helical tube (5) in this example had the following dimensions:
Internal diameter: dᵢ=7 mm
Diameter of curvature: D=250 mm
Pitch: h=40 mm
Dimensionless pitch: b=0.028
Dimensionless ratio of curvature: a=0.16

The solution to be worked up, which had a 2-EHA concentration of 52.5% by weight and included high boilers such as polymers and catalyst, was supplied to a preheater (2) operated with Marlotherm SH and heated. Preheating was at 130° C. The heated solution was discharged from the preheater via a conduit. The absolute pressure in the preheater was adjusted to 1.5 bar by a downstream pressure-maintenance device (3), which was designed as a shut-off valve having an internal diameter of 10 mm. A conventional shell-and-tube apparatus having a heat-transfer surface area of 0.1 m² served as the preheater. Downstream of the pressure-maintenance device (3), the heated solution was depressurized to an absolute pressure of 0.5 bar and supplied to the helical-tube evaporator (5) at a temperature of 120° C.

The absolute pressure in the separator (6) was 20 mbar. The feed rate of mixture (1) was 3 kg/h. The temperature in the separator (6) was 150° C. The evaporation rate achieved during the experiment was 68%.

The composition of the liquid mixture (1) flowing into the helical-tube evaporator (4) was as in comparative example 1:

| | | |
|---|---|---|
| Water | 0.4% | by weight |
| 2-Ethylhexene isomers | 0.1% | by weight |
| Acrylic acid | 0.4% | by weight |
| 2-Ethylhexanol | 0.9% | by weight |
| 2-Ethylhexyl acrylate | 52.5% | by weight |
| 2-Ethylhexyl 3-(2-ethylhexoxy)-propionate | 28.8% | by weight |
| 2-Ethylhexyl 2-diacrylate | 5.8% | by weight |
| p-Toluenesulfonic acid | 4.1% | by weight |
| Additional components and polymers | 7.0% | by weight |

The distillate (9) of 2.04 kg/h had the following composition:

| | | |
|---|---|---|
| Water | 0.2% | by weight |
| 2-Ethylhexene isomers | 2.6% | by weight |
| Acrylic acid | 0.2% | by weight |
| 2-Ethylhexanol | 2.0% | by weight |
| 2-Ethylhexyl acrylate | 70.2% | by weight |
| 2-Ethylhexyl 3-(2-ethylhexoxy)-propionate | 17.2% | by weight |
| 2-Ethylhexyl 2-diacrylate | 5.2% | by weight |
| p-Toluenesulfonic acid | 2.0% | by weight |
| Additional components and polymers | 0.4% | by weight |

The residue (10) of 0.96 kg/h had the following composition:

| | | |
|---|---|---|
| Water | 0.1% | by weight |
| 2-Ethylhexene isomers | 0.1% | by weight |
| Acrylic acid | 0.3% | by weight |
| 2-Ethylhexanol | 0.9% | by weight |
| 2-Ethylhexyl acrylate | 8.0% | by weight |
| 2-Ethylhexyl 3-(2-ethylhexoxy)-propionate | 40.0% | by weight |
| 2-Ethylhexyl 2-diacrylate | 5.0% | by weight |
| p-Toluenesulfonic acid | 25.0% | by weight |
| Additional components and polymers | 20.6% | by weight |

Compared to the existing workup process from the prior art, which is described in example 2, the process according to the invention using the helical-tube evaporator allowed the amount of residue (10) to be reduced from 0.42 kg per kg feed to 0.32 kg per kg feed.

Moreover, in example 2, the cleavage that occurs in the existing workup process resulted in the formation of a larger amount of 2-ethylhexene isomers.

Compared to the existing workup process, the process according to the invention using the helical-tube evaporator allowed the amount of 2-ethylhexene isomers to be reduced from 0.12 kg per kg feed to 0.02 kg per kg feed.

Irreversible coating of the heated surfaces of the helical-tube evaporator was not observed even after several days of operation.

### Comparative Example 1

Comparative example 1 describes a discontinuous process configuration according to the prior art and is elucidated in more detail below with reference to FIG. 3.

The separation of the high boilers, which are for example polymers, was carried out in a stirred tank (13) operated discontinuously with external heating, the heating being effected via heating steam (14). The stirred tank had a volume of 8 m³. The amount of mixture (1) as feed was 6 tonnes at a temperature of 120° C. The absolute pressure in the stirred tank (12) was set at 40 mbar. The temperature in the bottoms region of the stirred tank (12) was 145° C.

The heating of the stirred tank was switched off after 10 hours.

The vapor stream from the stirred tank was condensed in the condenser (12), which was designed as a conventional shell-and-tube heat exchanger having a heat exchange surface area of 100 m².

The distillate (9) was recycled back to the process. In the process, the unwanted 2-ethylhexene isomers obtained as low boilers were then removed and incinerated.

The composition of the mixture (1) flowing into the stirred tank was as in example 1:

| | | |
|---|---|---|
| Water | 0.4% | by weight |
| 2-Ethylhexene isomers | 0.1% | by weight |
| Acrylic acid | 0.4% | by weight |
| 2-Ethylhexanol | 0.9% | by weight |
| 2-Ethylhexyl acrylate | 52.5% | by weight |
| 2-Ethylhexyl 3-(2-ethylhexoxy)-propionate | 28.8% | by weight |
| 2-Ethylhexyl 2-diacrylate | 5.8% | by weight |
| p-Toluenesulfonic acid | 4.1% | by weight |
| Additional components and polymers | 7.0% | by weight |

The distillate (9) of 4400 kg had the following composition:

| | | |
|---|---|---|
| Water | 0.7% | by weight |
| 2-Ethylhexene isomers | 16.0% | by weight |
| Acrylic acid | 1.3% | by weight |
| 2-Ethylhexanol | 14.0% | by weight |
| 2-Ethylhexyl acrylate | 70.0% | by weight |
| 2-Ethylhexyl 3-(2-ethylhexoxy)-propionate | 2.3% | by weight |
| 2-Ethylhexyl 2-diacrylate | 0.7% | by weight |
| p-Toluenesulfonic acid | 0.1% | by weight |
| Additional components and polymers | 0.9% | by weight |

Cleavage resulted in the formation of 704 kg of 2-ethylhexene isomers per batch process. Based on the feed rate, the amount of 2-ethylhexene isomers formed was 0.12 kg per kg of feed.

The residue (10) of 1600 kg had the following composition:

| | | |
|---|---|---|
| Water | 0.1% | by weight |
| 2-Ethylhexene isomers | 0.1% | by weight |
| Acrylic acid | 0.3% | by weight |
| 2-Ethylhexanol | 0.9% | by weight |
| 2-Ethylhexyl acrylate | 21.0% | by weight |
| 2-Ethylhexyl 3-(2-ethylhexoxy)-propionate | 20.0% | by weight |
| 2-Ethylhexyl 2-diacrylate | 5.0% | by weight |
| p-Toluenesulfonic acid | 24.0% | by weight |
| Additional components and polymers | 28.6% | by weight |

To improve the pumpability of the residue (10), the residue (10) was mixed with 900 kg of Oxo Oil 9N and subsequently thermally utilized.

The total amount of residue was 2500 kg; based on the feed the amount of residue was 0.42 kg/kg.

After a few days of operation, the stirred tank needed to be cleaned because of soiling. The polymers that form contaminate the inner wall of the stirred tank, which also serves as a heat-transfer surface, and this meant that the heat transfer necessary for evaporation was no longer possible.

1.-18. (canceled)

19. A continuous process for obtaining 2-ethylhexyl acrylate (2-EHA) from a mixture that is liquid under an absolute pressure in the range from 0.5 to 100 bar and has a temperature in the range from 0 to 300° C., comprising 2-EHA, at least one high boiler, at least one homogeneous catalyst, and at least one low boiler, comprising
depressurizing the mixture is by a pressure-maintenance device to an absolute pressure level in the range from 0.1 to 10 bar, wherein a resulting two-phase gas/liquid mixture is continuously supplied to a helical-tube evaporator in which, at a temperature in the range from 50 to 300° C., the 2-EHA content of the liquid phase of the two-phase gas/liquid mixture is reduced by partial evaporation, being accompanied by a parallel increase in the 2-EHA content of the gas phase of the two-phase gas/liquid mixture, and
discharging the two phases in the form of a resulting two-phase gas/liquid output stream.

20. The process according to claim 19, wherein a preheater upstream of the pressure-maintenance device heats the liquid mixture to a temperature in the range from 100 to 200° C., if the mixture does not have a temperature of at least 100° C.

21. The process according to claim 19, wherein the helical-tube evaporator is operated at an absolute pressure in the range from 1 to 2000 mbar.

22. The process according to claim 19, wherein the proportion of 2-EHA in the liquid phase is in a single pass through the helical-tube evaporator reduced to a 2-EHA content of less than 20% by weight.

23. The process according to claim 19, wherein the proportion of 2-EHA in the liquid phase is in a single pass through the helical-tube evaporator reduced to a 2-EHA content of less than 10% by weight.

24. The process according to claim 19, wherein the formation of 2-ethylhexene isomers in the process is less than 2% by weight based on the mixture.

25. The process according to claim 19, wherein part of the liquid phase of the two-phase gas/liquid output stream withdrawn from the helical-tube evaporator (4) is returned to the helical-tube evaporator for further partial evaporation.

26. The process according to claim 19, wherein a stripping gas is added to the two-phase gas/liquid mixture downstream of the pressure-maintenance device, so that the partial evaporation in the helical-tube evaporator is carried out in the presence of a stripping gas.

27. The process according to claim 19, wherein, instead of a single helical-tube evaporator, two or more helical-tube evaporators are connected in series to form an evaporator cascade, wherein the gas/liquid mixture flowing into the evaporator cascade undergoes a gradual reduction in the 2-EHA content of its liquid phase through partial evaporation of the liquid phase.

28. The process according to claim 19, wherein, instead of a single helical-tube evaporator, two or more helical-tube evaporators are connected in parallel to form an evaporator cascade, wherein the gas/liquid mixture flowing into the evaporator cascade undergoes a reduction —split between the two evaporators—in the 2-EHA content of its liquid phase through partial evaporation of the liquid phase.

29. The process according to claim 27, wherein the individual helical-tube evaporators of the evaporator cascade are operated at different or identical pressures in the range from 1 to 2000 mbar.

30. The process according to claim 29, wherein the individual helical-tube evaporators of the evaporator cascade are operated at least partially with heat integration.

31. The process according to claim 19, wherein the two-phase gas/liquid output stream from the helical-tube evaporator is supplied to a downstream separator that is operated at an absolute pressure in the range from 1 to 2000 mbar.

32. The process according to claim 19, wherein the two-phase gas/liquid output stream from the helical-tube evaporator is supplied to a downstream separator that is operated at an absolute pressure in the range from 5 to 200 mbar.

33. The process according to claim 31, wherein the downstream separator is a gravity separator.

34. The process according to claim 31, wherein the formation of polymers in the helical-tube evaporator and in the separator is together less than 5% by weight based on the mixture.

35. The process according to claim 31, wherein the gaseous fraction of the two-phase gas/liquid output stream supplied to the separator is supplied from the separator to a condenser and condensed in the condenser to form a distillate.

36. The process according to claim 19, wherein the helical tube in the helical-tube evaporator, or each individual helical tube of a helical-tube evaporator in the case of an evaporator cascade, independently has a dimensionless ratio of curvature a in the range from 0.01 to 0.5 and a dimensionless pitch b in the range from 0.01 to 1.0.
